(19) United States
(12) Patent Application Publication
(10) Pub. No.: US 2003/0092073 A1
(43) Pub. Date: May 15, 2003

(54) Method for sequence specific biotinylation

(76) Inventors: Dorothee Ambrosius, Muenchen (DE); Martin Lanzendoerfer, Muenchen (DE); Michael Schraeml, Muenchen (DE); Manfred Watzele, Weilheim (DE)

Correspondence Address: HOFFMANN-LA ROCHE INC., PATENT LAW DEPARTMENT, 340 KINGSLAND STREET, NUTLEY, NJ, 07110

(21) Appl. No.: 10/251,313

(22) Filed: Sep. 20, 2002

(30) Foreign Application Priority Data
Sep. 25, 2001 (EP) 01122554.7
Dec. 13, 2001 (EP) 01129681.1

(51) Int. Cl. G01N 33/53; C12P 21/06

(52) U.S. Cl. 435/7.5; 435/68.1

(57) ABSTRACT

A method of preparing a biotinylated polypeptide in a cell-free peptide synthesis reaction mixture by contacting, under suitable conditions, a polypeptide to be biotinylated, with a reaction mixture that includes ribosomes, tRNA, ATP, GTP, nucleotides, biotin and amino acids, and a polypeptide that includes an enzymatically active domain of a BirA enzyme. The polypeptide to be biotinylated includes a BirA substrate sequence tag, and the polypeptide to be biotinylated and the polypeptide comprising an enzymatically active domain of a BirA enzyme, are expressed in situ in the reaction mixture, by at least one nucleic acid molecule encoding the polypeptide to be biotinylated, and the enzymatically active domain of a BirA enzyme, respectively.

### FIELD OF THE INVENTION

The invention relates to an improved method for sequence specific biotinylation of polypeptides.

### BACKGROUND OF THE INVENTION

The enzyme biotin haloenzyme synthetase of Eschericia coli (“E. coli”), is a biotin ligase (hereinafter also referred to as “BirA”) that is the product of the qrA gene (Cronan, J. E., Jr., Cell 58 (1989) 427-429). BirA catalyzes the covalent addition of biotin, in vivo, to the ε-amino group of a lysine side chain in its natural substrate, biotin carboxyl carrier protein (“BCCP”) (Cronan, J. E., Jr., et al., J. Biol. Chem. 265 (1990) 10327-10333). BCCP is a subunit of acetyl-CoA carboxylase and, in E. coli, BCCP is biotinylated. Biotinylation of proteins using a biotinylation enzyme by recombinant means is described, e.g., in WO 95/04069, incorporated by reference herein.

Sequence specific enzymatic biotinylation, (also referred to herein as “specific biotinylation” or preparing, “specifically biotinylated” polypeptides) using BirA is also described for recombinant polypeptides during expression in E. coli (Tsao, K.-L., et al., Gene 169 (1996) 59-64), incorporated by reference herein. Altman, J. D., et al., Science 274 (1996) 94-96, incorporated by reference herein, describe the enzymatic biotinylation of isolated polypeptides in vitro, using also BirA. However, such a method is very laborious, requiring considerably more purification steps compared to conducting the biotinylation in vivo. First, the protein must be prepared, isolated and purified. Subsequently, biotinylation is performed, and thereafter, another purification is carried out. Parrott, M. B., and Barry, M. A., in Biochem. Biophys. Res. Communications 281 (2001) 993-1000, incorporated by reference herein, describe the metabolic biotinylation of secreted and cell-surface proteins from mammalian cells using the endogenous biotin ligase enzymes of the mammalian cell. Saviranta, P., et al., in Bioconjug. Chem. 9 (1998) 725-735, incorporated by reference herein, describe the in vitro enzymatic biotinylation of recombinant Fab fragments through a peptide acceptor tail. The proteins were recombinantly produced in E. coli, purified and subsequently biotinylated in vitro with BirA. After the removal of non-biotinylated Fab fragments, the overall yield of biotinylated Fab was 40%.

Both the in vitro as well as the in vivo biotinylation of heterologous polypeptides using biotin ligases such as BirA suffer from several drawbacks. In vitro biotinylation, i.e., biotinylation in a cell free reaction medium, is very time-consuming and laborious, and in vivo biotinylation, i.e., taking place within host cells such as E. coli, results in products containing considerable amounts of BCCP. In a particular drawback, biotinylated BCCP is produced by E. coli during the in vivo methods, and the biotinylated BCCP cannot be completely removed from the desired biotinylated polypeptides.

Accodingly, there has been a need for an improved and simplified method for the specific enzymatic biotinylation of polypeptides to produce specifically biotinylated polypeptides of high purity, high activity and high yield.

In addition, there has been a need for an in vitro method for the specific enzymatic biotinylation of polypeptides that requires only a single in vitro reaction medium to produce specifically biotinylated polypeptides of high purity, high activity and high yield.

### SUMMARY OF THE INVENTION

Accordingly, the invention provides methods for the synthesis of specifically biotinylated polypeptides. The invention also provides for specifically biotinylated polypeptides having activity that is higher than the activity found for such polypeptides biotinylated in an in vivo cell fermentation system.

The inventive method is preferably conducted in vitro, i.e., in a cell free or extracellular reaction mixture. In particular, the synthesis is performed in a cell-free peptide synthesis reaction mixture that includes a ribosome-containing cell lysate of a prokaryotic or eukaryotic cell, by translation or transcription/translation of a nucleic acid encoding the polypeptide, whereas the reaction mixture contains biotin and a protein having BirA enzyme activity.

Thus, methods are provided for producing a specifically biotinylated polypeptide. The inventive methods include contacting, under suitable conditions, a polypeptide to be biotinylated with a reaction mixture that comprises ribosomes, tRNA, ATP, GTP, nucleotides, biotin and amino acids, and a polypeptide that includes an enzymatically active domain of a BirA enzyme, wherein the polypeptide to be biotinylated includes a BirA substrate sequence tag. The reaction mixture is preferabbly a ribosome-containing cell lysate of a prokaryotic source, e.g., from E. coli.

More preferably, both the polypeptide to be biotinylated and the polypeptide comprising an enzymatically active domain of a BirA enzyme are expressed in situ in the reaction mixture, by at least one nucleic acid molecule encoding the polypeptide to be biotinylated, and encoding the enzymatically active domain of a BirA enzyme, respectively. In an alternative option, the polypeptide to be biotinylated and/or the polypeptide with the enzymatically active domain of a BirA enzyme are expressed in situ by two diferent nucleic acids, e.g., any art-known expression vector compatible with the respective polypeptides selected reaction mixture.

It will be appreciated that the BirA substrate sequence tag is optionally located at any position in the polypeptide to be biotinylated, allowing for selective biotinylation, ie., sequence specific biotinylation of a polypeptide. Preferably, the BirA substrate sequence tag located at either the N-terminal or the C-terminal of the polypeptide to be biotinylated.

Optionally, the biotinylated polypeptide is bound to a biotin-binding surface as it is produced, and/or at the conclusion of the biotinylation reaction, and then directly employed in bound or soluble form in any suitable art-known assay, or other reaction procedure requiring a particular polypeptide to be present in biotinylated form.

Preferably, the polypeptide to be biotinylated is expressed in situ as a fusion protein that includes a polypeptide of interest, e.g., any polypeptide that it is desirable to link to biotin, and any art-known BirA substrate sequence tag. Many such BirA substrate sequence tags are known. Preferrably, the BirA substrate sequence tag includes an Ala Met Lys Met motif (SEQ ID NO: 14). More preferably, the BirA substrate sequence tag has a peptide sequence selected from the group consisting of SEQ ID NO. 1, SEQ ID NO: 2, SEQ ID NO: 3, SEQ ID NO: 4, SEQ ID NO: 5, SEQ ID NO: 6 or SEQ ID NO: 7.

Suitable reaction conditions for the inventive methods include, e.g., a temperature from about 20° C. to to about 36° C. Generally, the reaction generally takes from about 10 to to about 30 hours to produce a desired quantity of biotinylated protein.

### DETAILED DESCRIPTION OF THE INVENTION

The invention will now be described in terms of its preferred embodiments. These embodiments are set forth to aid in understanding the invention but are not to be construed as limiting.

The invention provides improved methods for the production of a polypeptide (“polypeptide of interest”) that is specifically biotinylated at an N- or C-terminal sequence tag by site-specific enzymatic biotinylation. Preferably, the inventive biotinylation methods are conducted in vitro, and more prefereably, in a single process step. The polypeptide to be biotinylated can be of any size or molecular weight that is required. Preferably, the polypeptide of interest has a molecular weight of about 8 kDa to about 120 kDa, and/or a length of about 100 to about 400 amino acid residues. According to the invention it is surprisingly possible to produce such biotinylated polypeptides without substantial contamination by biotin carboxyl carrier protein (“BCCP”), and without the need for intermediate isolation of the non-biotinylated polypeptide if a cell-free peptide synthesis reaction mixture is used for the polypeptide synthesis.

Broadly, the methods of the invention include expressing a nucleic acid vector in a cell free peptide synthesis reaction mixture, wherein the reaction mixture includes elements necessary for peptide sysnthesis, e.g., ribosomes, tRNA, ATP, GTP, nucleotides, and amino acids, and wherein the nucleic acid expresses a polypeptide to be biotinylated. The method is contemplated as a single step method, although for greater clarity the methods may be described as follows.

Expressing a first nucleic acid in the cell free reaction mixture to produce a polypeptide that includes a BirA substrate sequence tag, ie., a fusion protein that serves as a substrate for an enzyme having BirA activity. Expressing, simultaneously or in any order relative to expression of the first nucleic acid, a second nucleic acid in the reaction mixture, to produce a polypeptide that includes an enzymatically active domain of a BirA enzyme. The first and second nucleic acids can be the same or different, e.g., the respective polypeptides can be expressed by a single nucleic acid molecule and/or by two or more separate nucleic acid molecules. As substrate and enzyme accumulate in the presence of biotin in the cell free reaction mixture, the fusion protein having a BirA substrate sequence tag is biotinylated.

Optionally, the inventive method includes isolating the biotinylated polypeptide from the reaction mixture by any art known method. Alternatively, the biotinylated polypeptide is employed in situ by simply incubating the reaction mixture with a support having a biotin-binding surface, e.g., a surface that includes immobilized avidin or streptavidin, under such conditions that the biotinylated polypeptide is bound to the biotin binding surface.

Such surfaces include, simply by way of example, surfaces of art-known supports such as beads, plates, cuvettes, filters, titer plates, PCR plates, and the like, that have avidin, streptavidin and/or any art known derivative of these agents linked or coated to the surface(s) of those supports. The supports are generally made of conventional materials, e.g., plastic polymers, cellolose, glass, ceramic, stainless steel alloy, and the like.

A “cell-free peptide synthesis reaction mixture” according to the invention is art-known and is a cell-free lysate of prokaryotic or eukaryotic cells that includes, e.g., ribosomes, tRNA, ATP, GTP, nucleotides, and amino acids. A preferred prokaryote source of the lysate is E. coli.

Cell-free polypeptide synthesis is well known. In 1988 Spirin et al. developed a continuous-flow cell-free (“CFCF”) translation and coupled transcription/translation system in which a relatively high amount of protein synthesis occurs (Spirin, A. S., et al., Science 242 (1988) 1162-1164, incorporated by reference herein). For cell-free protein synthesis, cell lysates containing ribosomes were used for translation or transcription/translation. Cell-free extracts from E. coli were developed by, for example, Zubay, G., Ann. Rev. Genetics 7 (1973) 267-287 and were used by Pratt, J. M., et al., Nucleic Acids Research 9 (1981) 4459-4479 and Pratt et al., Transcription and Translation: A Practical Approach, Hames and Higgins (eds.), pp. 179-209, IRL Press, 1984, all of which are incorporated by reference herein. Further developments of cell-free protein synthesis are described in U.S. Pat. No. 5,478,730; U.S. Pat. No. 5,571,690; EP 0 932 664; WO 99/50436; WO 00/58493; and WO 00/55353, all of which are incorporated by reference herein.

Eukaryotic cell-free expression systems are described, for example, by Skup, D., and Millward, S., Nucleic Acids Research 4 (1977) 3581-3587; Fresno, M., et al., Eur. J. Biochem. 68 (1976) 355-364; Pelham, H. R., and Jackson, R. J., Eur. J. Biochem. 67 (1976) 247-256; and in WO 98/31827, all of which are incorporated by reference herein.

As noted above, holocarboxylase synthetase (also art-known as EC6.3.4.15, biotin protein ligase (“BPL” or “BirA”) is an enzyme responsible for the covalent attachment of biotin to the cognate protein. Biotin ligase is highly substrate specific and biotinylates only proteins showing a very high degree of conservation in the primary structure of the biotin attachment domain. This domain preferably includes the highly conserved AMKM (SEQ ID NO: 14) tetrapeptide reported, e.g., by Chapman-Smith, A., and Cronan, J. E., Jr., J. Nutr. 129, 2S Suppl., (1999) 477S-484S). Recombinant BirA enzyme is described in WO 99/37785. Both references are incorporated by reference herein.

Biotin ligase activity is defined by the manufacturer (Avidity, Inc. of Denver Colo.) as follows: 1 Unit of BirA activity is the amount of enzyme that will biotinylate 1 pmol of peptide substrate in 30 min at 30° C. using the reaction mixture containing peptide substrate at 38 μM. Avidity Inc. reports that the peptide substrate was a 15-mer variant of Sequence No. 85 as identified by Schatz, P. J., Biotechnology 11 (1993) 1138-1143, incorporated by reference herein.

BirA can be added to the reaction mixture as protein, or can be added as a nucleic acid (encoded by an expression vector, e.g., RNA, DNA) which is expressed (transcribed/translated) in a reaction system, as is the polypepide or protein of interest. If already added as a protein, it is preferably used in an amount of about 10,000 to 15,000 units, or more preferably 12,500 units, added to a volume of 1 ml, and/or as expressed in situ in the reaction mixture, as exemplified herein. The amount of nucleic acid depends on the expression rate of the vector and the amount of BirA enzyme required in the reaction mixture. 1 ng of BirA plasmid DNA (e.g. on the basis of a commercially available E. coli expression vector such as pIVEX® vectors, (Roche Applied Science, Indianapolis, Ind.), or even less, is sufficient for a quantitative biotinylation reaction of proteins fused with a BirA biotinylation substrate peptide. The maximum yield of expressed and specifically biotinylated fusion protein is achieved when the desired target protein encoding plasmid DNA is added at 10-15 μg and the plasmid DNA, that is responsible for the coexpression of BirA, is introduced in an amount between 1-10 ng. The ratio of fusion protein encoding plasmid-DNA to BirA encoding plasmid DNA was found to be optimal at a ratio of about 1500:1. It was found that the same level as above is sufficient for quantitative biotinylation of the expressed fusion protein. D(+)-biotin was to the reaction mixture in a concentration ranging from 1 to 10 μM, and more preferably at a concentration of about 2 μM.

The polypeptide of interest, i.e., the polypeptide to be specifically biotinylated, includes a peptide sequence that is recognized by the biotin protein ligase, i.e., a BirA substrate sequence tag. The BirA substrate sequence tag is preferably located at the N-terminus or C-terminus of the polypeptide of interest.

Unless otherwise specified, a BirA substrate sequence tag is defined herein as a peptide sequence present in a polypeptide that provides a specific site for BirA to biotinylate the polypeptide substrate. Many BirA substrate sequence tags are known to the art. As already mentioned, such sequences exhibit a common structure, which preferably contains the amino acid motif AMKM (SEQ ID NO: 14) or certain variations thereof. In addition, there exist peptide sequences which do not contain this consensus sequence, but can also be biotinylated by biotin protein ligases (Schatz, P. J., Biotechnology 11 (1993) 1138-1143, incorporated by reference herein). Such sequences function as BirA substrate sequence tags, and preferably have a length of about less than 50 amino acids, and most preferably a length of about 10 to 20 amino acids. Numerous specific and general examples are described in U.S. Pat. No. 6,265,552, incorporated by reference herein.

Preferred BirA substrate sequence tags are described in U.S. Pat. No. 6,265,552, and include SEQ ID NOs. 1-12 and 14-89 of that patent. More preferred are BirA substrate sequence tag™ (Avidity, Inc., Indianapolis, Ind.) and PINPOINT™ (Promega Corporation, Madison, Wis.) that are exemplified herein.

Additional examples of polypeptide sequences which can be biotinylated enzymatically and site-specifically are also described in Cronan, J. E., Jr., et al., J. Biol. Chem. 265 (1990) 10327-10333; and Samols, D., et al., J. Biol. Chem. 263 (1988) 6461-6464, all of which are incorporated by reference herein. These examples are shown herein by SEQ ID NO:1 to SEQ ID NO:7. Further examples are shown in U.S. Pat. Nos. 5,723,584; 5,874,239; and 5,932,433, all of which are incorporated by reference herein.

After the expression of the fusion polypeptide in the cell-free system, biotinylation occurs under standard reaction conditions, preferably within 10 to 30 hours at 20° C. to 36° C., most preferably at about 30° C., and the reaction mixture is preferably dialyzed for concentration and buffer exchange, and then centrifuged.

In a preferred embodiment of the invention, the solution is, due to its high purity, directly used for immobilization of the biotinylated polypeptide on surfaces which contain immobilized avidin or streptavidin (e.g. microtiter plates or biosensors) without further purification. According to the invention it is possible to produce highly pure biotinylated polypeptides which can be bound to surfaces in ligand binding experiments, e.g. surface plasmon resonance spectroscopy or ELISA assays.

Optionally, biotinylated polypeptides produced according to the present invention can be further purified, as needed, under native conditions using matrices containing immobilized (preferably monomeric) avidin, streptavidin, or derivatives thereof.

It is also contemplated that modified forms of avidin or streptavidin are employed to bind or capture polypeptides biotinylated by the methods of the invention. A number of modified forms of avidin or streptavidin that bind biotin specifically, but with weaker affinity to facilitate a one step purification procedure are known. Such modified forms of avidin or streptavidin include, e.g., physically modified forms (Kohanski, R. A. and Lane, M. D. (1990) Methods Enzymol. 194-200), chemically modified forms such as nitro-derivatives (Morag, E., et al., Anal. Biochem. 243 (1996) 257-263) and genetically modified forms of avidin or streptavidin (Sano, T., and Cantor, C. R., Proc. Natl. Acad. Sci.USA 92 (1995) 3180-3184, and all of the foregoing references are incorporated by reference herein).

The following examples, references, sequence listing and figures are provided to aid the understanding of the present invention, the true scope of which is set forth in the appended claims. It is understood that modifications can be made in the procedures set forth without departing from the spirit of the invention.

### DESCRIPTION OF THE FIGURES

FIG. 1 illustrates a comparison of the biotinyl fusion proteins AVITAG™-PEX2 and PINPOINT™-PEX2. Two Western blots are shown, where biotinylated protein was detected with streptavidin peroxidase (“SA-POD”) conjugate in monomer avidin-sepharose elution fractions.

FIG. 1, left panel, illustrates RTS® 500 AVITAG™-PEX2: Lane 1: dialyzed and centrifuged supernatant of RTS 500 extract applied to the column. A second band under the target band indicates proteolytic degradation. Lane 2: column wash. Lanes 3, 4 and 5: fractions of the 5 mM biotin elution peak.

FIG. 1, right panel, illustrates E. coli PINPOINT™-PEX2: Lane 1: centrifuged supernatant of E. coli cell lysate applied to the column. Lane 2: column wash. Lanes 3-8: fractions of the elution peak containing PINPOINT™-PEX2, whereby the co-concentration of a proteolytic degradation product of BCCP becomes obvious in lane 5 and lane 6 [3,16]. Lane 9: pooled elution fractions after ultrafiltration.

FIG. 2 illustrates a Streptavidin-POD Western blot showing biotinylated AVITAG™-PEX2 fusion protein.

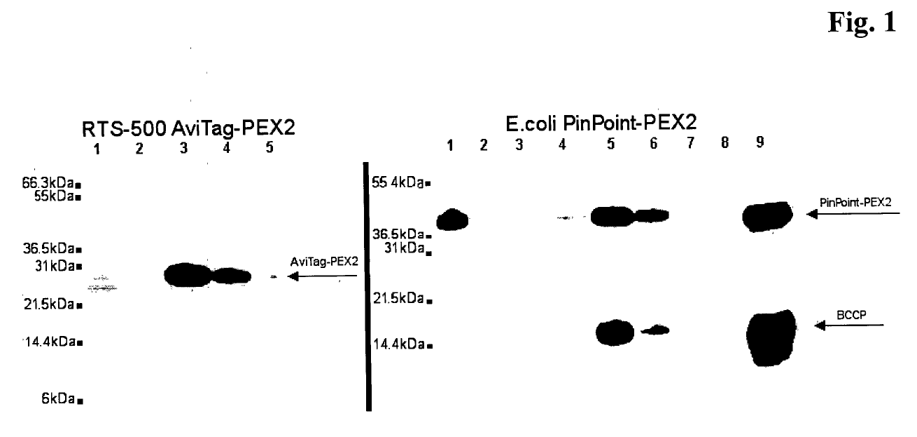

The biotinylation reaction was done by coexpression of BirA. As a positive control chemically biotinylated PEX2 was used. Lanes 1-4 show the following:

[1] 10 μg pIVEX2.1MCS AVITAG PEX2, 2 μM biotin, no pIVEX2.1MCSBirA addition.

[2] 330 ng chemically biotinylated PEX2, positive control.

[3] 13 ng chemically biotinylated PEX2, positive control.

[4] 7 ng chemically biotinylated PEX2, positive control not detectable.

[5] 10 μg pIVEX2.1MCS AVITAG PEX2, 2 μM biotin, 1 μg pIVEX2.1MCSBirA

[6] 10 μg pIVEX2.1MCS AVITAG PEX2, 2 μM biotin, 100 ng pIVEX2.1MCSBirA

[7] 10 μg pIVEX2.1MCS AVITAG PEX2, 2 μM biotin, 10 ng pIVEX2.1MCSBirA.

[8] 10 μg pIVEX2.1MCS AVITAG PEX2, 2 μM biotin, 1 ng pIVEX2.1MCSBirA.

### DESCRIPTION OF SEQUENCES AND SEQUENCE NUMBERS

SEQ ID NO: 1 1.3S transcarboxylase subunit of Propionibacterium shermanii

SEQ ID NO: 2 BCCP E. coli

SEQ ID NO: 3 Biotinylation peptide originating from the 1.3S transcarboxylase subunit

SEQ ID NO: 4 Biotinylation peptide AAW46671

SEQ ID NO: 5 Biotinylation peptide AAW46656

SEQ ID NO: 6 AVITAG™ Biotinylation peptide

SEQ ID NO: 7 PINPOINT™ Biotinylation peptide

SEQ ID NO: 8 Primer

SEQ ID NO: 9 Primer

SEQ ID NO: 10 Primer

SEQ ID NO: 11 Primer

SEQ ID NO: 12 Primer

SEQ ID NO: 13 Primer

SEQ ID NO: 14 Biotinylation peptide motif.

### EXAMPLE 1

### Expression of PINPOINT™-PEX2 in vivo—E. coli Comparison

PEX2 is the non-catalytic C-terminal hemopexin-like domain of matrix metalloproteinase 2 (MMP-2) (Brooks, P. C., et al., Cell 92 (1998) 391-400).

The encoding gene was amplified by PCR using the sense primer 5′-ATA AGA ATA AGC TTC CTG AAA TCT GCA AAC AGG ATA TCG-3′ (SEQ ID NO:8) and antisense primer ‘5’-ATA GTT TAG CGG CCG CTT ATC AGC CTA GCC AGT CG-3′ (SEQ ID NO:9). PCR was performed in 30 cycles with a temperature profile as follows: 1 min at 94° C., 1 min at 48° C. and 1 min at 72° C. The PCR product was cloned as a NotI/HindIII fragment into an isopropyl-β-D-thiogalactopyranoside (IPTG)-inducible E. coli expression vector. The plasmid was transformed into an E. coli strain which contained the helper plasmid pUBS520 (Brinkmann, U., et al., Gene 85 (1989) 109-114). Cells were grown in LB-media containing 2 μM biotin, 100 μg/ml ampicillin and 50 μg/ml kanamycin. An overnight culture was used to inoculate 1 l medium of the same composition, which was incubated under vigorous shaking at 37° C. At OD595=0.5, expression was induced with 1 mM IPTG for 5 h. The cells (2.500 g) were harvested by centrifugation. The cell paste was resuspended (5 ml/g cell paste) in 50 mM TRIS pH 7.2, 20 mM NaCl, 5 mM CaCl2, 1 mg/ml lysozyme, Complete EDTA-Free protease inhibitor cocktail (Roche Diagnostics GmbH, Penzberg, Germany) and subsequently incubated for 20 min at room temperature. Further cell lysis was performed by sonication on ice until the suspension was no longer viscous. Crude lysate was centrifuged at 10,000 g for 30 min at 4° C. and the supernatant was filtered through a 0.22 μm filter.

### EXAMPLE 2

### Expression of AVITAG™-PEX2 in the Cell-Free Expression System

The PEX2 gene was genetically fused with AVITAG™ coding DNA by add-on PCR using the primers

| | | |
|---|---|---|
| 5′-GAAGGCATATGGGTCTGAACG-3 | (25 pmol) | (SEQ ID NO:10) |

′,

| | | |
|---|---|---|
| 5′-CTCAGAAAATCGAATGGCACGAA | (10 pmol) | (SEQ ID NO:11) |
| GCGACCCTGAAATCTGCAAACAGG-3 | | |

′,

| | | |
|---|---|---|
| 5′-GCCATTCGATTTTCTGAGCTTCG | (10 pmol) | (SEQ ID NO:12) |
| AAGATGTCGTTCAGACCCATATGCC- | | |

3′ and

| | | |
|---|---|---|
| 5′-GCCGCTCGAGTCAGCAGCCTAGC | (25 pmol) | (SEQ ID NO:13) |

CAGTCGG-3′.

The PCR program was performed as described above. The PCR product was digested with NdeI and XhoI and was cloned into an E. coli expression plasmid (pIVEX®2.3MCS plasmid of cocktail (Roche Diagnostics GmbH, Penzberg, Germany), previously cut with the same restriction enzymes. The plasmid was propagated in E. coli and isolated. 15 μg plasmid-DNA (ratio 260 nm/280 nm>1.8) and 12.500 units biotin ligase holoenzyme (˜2,5 ug biotin ligase, EC 6.3.4.15; Avidity Inc., Denver, USA) were added to the reaction mixture (1 ml) of a commercially available cell-free expression system (Rapid Translation System, RTS®500, Roche Diagnostics GmbH, Penzberg, Germany). Biotin ligase activity is defined by the manufacturer as follows: 1 Unit of BirA is the amount of enzyme that will biotinylate 1 pmol of peptide substrate in 30 min at 30° C. using the reaction mixture containing peptide substrate at 38 μM. Avidity, Inc. states that the peptide substrate was a 15-mer variant of Sequence No. 85 as identified by Schatz, P. J., Biotechnology 11 (1993) 1138-1143. The commercial enzyme is dissolved or suspended in carrier at 1 mg/ml and has an activity of 5,000 units of activity per μg).

Biotin concentration was adjusted to 2 μM in the reaction mixture and the feeding solution (12 ml). Protein expression was performed in the RTS®500 Incubator under stirring (130 rpm) for 17 h at 30° C. The product solution was subsequently dialyzed against buffer W2 (see Example 3) and centrifuged at 10,000 g for 30 min at 4° C.

The E. coli lysate was prepared according to Zubay G., Ann. Rev. Genetics 7 (1973) 267-287, and dialyzed against a buffer containing 100 mM HEPES-KOH pH 7.6/30° C., 14 mM magnesium acetate, 60 mM potassium acetate, 0.5 mM dithiothreitol. The lyophilized lysate was solubilized as recommended in the RTS®500 system manual.

Reaction Mixture:

185 mM potassium acetate, 15 mM magnesium acetate, 4% glycerol, 2.06 mM ATP, 1.02 mM CTP, 1.64 mM GTP, 1.02 mM UTP, 257 μM of each amino acid (all 20 naturally occurring amino acids), 10.8 μg/ml folic acid, 1.03 mM EDTA, 100 mM HEPES-KOH pH 7.6/30° C., 1 μg/ml rifampicin, 0.03% sodium azide, 40 mM acetyl phosphate, 480 μg/ml tRNA from E. coli MRE600, 2 mM dithiothreitol, 10 mM mercaptoethane sulfonic acid, 70 mM KOH, 0.1 U/μl Rnase inhibitor, 15 μg/ml plasmid, 220 μl/ml E. coli A19 lysate, 2 U/μl T7-RNA polymerase.

Feeding Solution:

185 mM potassium acetate, 15 mM magnesium acetate, 4% glycerol, 2.06 mM ATP, 1.02 mM CTP, 1.64 mM GTP, 1.02 mM UTP, 257 μM of each amino acid (all 20 naturally occurring amino acids), 10.8 μg/ml folic acid, 1.03 mM EDTA, 100 mM HEPES-KOH pH 7.5/30° C., 1 μg/ml rifampicin, 0.03% sodium azide, 40 mM acetyl phosphate, 2 mM dithiothreitol, 10 mM mercaptoethane sulfonic acid, 70 mM KOH.

### EXAMPLE 3

### Purification and Quantification

Purification of Biotinylated Fusion Proteins:

1 ml monomeric avidin sepharose resin (SOFTLINK, Promega, Madison USA) was filled in a Pharmacia HR-5 column. After washing the column with 10 CV buffer W1 (50 mM TRIS pH 7.2, 20 mM NaCl) and 10 CV buffer W2 (W1+5 mM CaCl2), cell extract according to Example 1 or product solution according to Example 2 was applied with a flow rate of 0.1 ml/min. Washing with buffer W2 was done until no more protein was detectable in the flow-path of the column. To elute biotinylated protein, buffer W2+5 mM biotin was applied. The eluted protein peak was separated in 0.5 ml fractions. Fractions, containing biotinylated target protein, were pooled, free biotin was removed during ultrafiltration with buffer W2.

Detection and Quantification of the Fusion Proteins:

The soluble and insoluble protein fractions were resolved by SDS-PAGE (10% BIS-TRIS SDS-polyacrylamide gel) and either stained with Coomassie brilliant blue or transferred to a PVDF-membrane by using the semy-dry Multiphor II apparatus (Pharmacia Biotech, Uppsala, Sweden) for 70 min at 120 V and room temperature. After the transfer was completed, the membrane was blocked in phosphate buffered saline (PBS) plus 0.2% Tween 20 (PBS-Tween) and 5% (w/v) dry milk powder with gentle agitation at 4° C. PEX2 bound to the PVDF-membrane was detected with a PEX2-specific antibody, that was prepared by standard methods. The antibody stock solution was 1.47 mg/ml polyclonal rabbit anti-PEX2-IgG, directed against the whole molecule. The membrane was incubated for 1 hour at room temperature in PBS-Tween, 2.5% (w/v) dry milk powder, containing PEX2 antiserum (1:50.000 v/v) followed by three ten minute washes. The membrane was incubated for 1 hr in PBS-Tween+2.5% (w/v) dry milk powder with 1:50,000 anti-mouse/anti-rabbit-IgG-POD conjugate (Roche Diagnostics GmbH, Penzberg, Germany) followed by three ten minute washes in PBS-Tween. The Western blot was developed with the Chemiluminescence Western Blotting Kit (Mouse/Rabbit, Roche Diagnostics GmbH, Penzberg, Germany).

After the densitometric detection of PEX2 protein, the membrane was reGenerated for 10 min in 0.1 M NaOH and subsequently washed 3×10 min in PBS-Tween. The membrane was blocked and washed again as described above. Detection of biotinylated fusion protein was carried out by incubating the reGenerated membrane in a 1:4000 (v/v) dilution of streptavidin-POD conjugate (Roche Diagnostics GmbH, Penzberg, Germany) in PBS-Tween buffer+2.5% (w/v) dry milk powder, for 1 hour. After washing the membrane three times for 10 min with PBS-Tween, the Western blot was developed again. Biotinylation levels of the PEX2 fusion proteins were determined by comparison of densitometric data of the two detection steps.

Densitometric quantification of detected protein bands was performed by calibration using verified quantities of recombinant, chemically biotinylated PEX2 and the software ImageMaster 1D Prime 1D Elite (Amersham Pharmacia Biotech Europe GmbH, Freiburg, Germany).

Plasmon Resonance Spectroscopy:

Activity of the biotinylated PEX2 fusion proteins was measured using plasmon spectroscopy (BIACORE 3000 technology, BIAcore AB, Uppsala, SE), that was run under HBS-P-buffer. PEX2 fusion proteins were immobilized on streptavidin coated BIAcore-SA chips in a manner as recommended by the manufacturer. Various dilution steps of a 200 nM TIMP2 (tissue inhibitor of metalloproteinase-2, Yu, A. E., et al., Biochem. Cell Biol. 74 (1996) 823-831) stock solution (0.33 mg/ml in 1×PBS-buffer) in HBS-P buffer were used to measure kinetic data in accordance to the manufacturers instructions. TIMP2 was eluted from the chip with IMMUNOPURE Gentle Ag/Ab Elution buffer (Pierce Biotechnology, Inc., Rockford, Ill.).

### EXAMPLE 4

### Investigation of Purity

Biotinylated PINPOINT™-PEX2 in E. coli (Comparison)

PEX2, N-terminally fused with the PINPOINT™-tag, (Promega Corporation, Madison, Wis.) was expressed and specifically biotinylated in vivo in E. coli in accordance with Example 1. The fusion protein has a calculated molecular mass of 36 kDa. Protein identity was confirmed by N-terminal Edman degradation. Harvesting of 1 liter of fermentation culture resulted in 6 grams of bacteria (wet mass). A total yield of 0.4 mg fusion protein per gram cell paste was determined by densitometric quantification of Western blots performed using PEX2-specific antibodies. Approximately 10% of the target protein was enriched in the supernatant of the cleared cell lysate. Quantification of biotinylation yield was determined by comparing densitometric data as described in material and methods. Using streptavidin-POD (peroxidase) conjugate in a colorimetric assay, no other biotinylated protein could be detected in the crude cell lysate, whereas monomeric avidin affinity chromatography enriched a second biotinylated protein in the elution fractions.

Further analysis of the eluate using streptavidin-POD conjugate revealed two protein bands. The first band with an approximate mass of 40 kDa was the desired biotinylated fusion protein PINPOINT™-PEX2. The second protein with a size of approximately 16 kDa is BCCP, the only biotinylated protein found (naturally occuring) in E. coli. Contamination with this second biotinylated protein accounted for up to 50% of the total yield. The PINPOINT™-PEX2 containing elution fractions were pooled. The excess of free biotin was removed via ultrafiltration.

Two samples with different degree of purity were analyzed in surface plasmon resonance spectroscopy using BIAcore technology. The activity of an immobilized ligand is indicated by the maximum analyte binding capacity. For the following measurements, it is helpful to note that RU are the resonance units in a BIAcore assay. One RU is standardized as 1 pg/square mm on a BIAcore chip coated with Streptavidin.(Biacore AB—Europe Regional Office Biacore AB Stevanage Herts, United Kingdom).

First, activity of the partially purified protein concentrate was analyzed. 380 RU of PINPOINT™-PEX2 (ligand) were immobilized on a BIAcore SA-chip. Saturation of the protein ligand on the chips surface with TIMP2 (analyte) was reached at Rmax=61RU. Based on this data, a ligand binding activity of 26% was calculated. In a second setting, 664RU of biotinyl-protein were immobilized by injecting supernatant of dialyzed and cleared cell lysate in the flow-cell. Saturation with the analyte TIMP2 was reached at 61 RUmax and the calculated ligand binding activity was 15%. In both cases kinetic data of the TIMP2/PINPOINT™-PEX2 interaction were determined. An equilibrium constant of KD=1.5×10⁻¹⁰ M was calculated using a numeric Langmuir simulation model of a binary complex formation.

Biotinylated AVITAG™-PEX2

PEX2, N-terminally fused with the AVITAG™ biotin-acceptor sequence, was expressed and biotinylated in vitro in the RTS®500 in accordance with Example 2. Biotinylation was facilitated by adding 12,500 units of BirA-enzyme to the reaction mix. The expressed fusion protein has a molecular mass of 25 kDa and was detected by Western blotting, using the PEX2-specific antibody and streptavidin-POD conjugate. When compared to a molecular weight standard, the fusion protein shows an apparent mass of 25 kDa in a 10% Bis-Tris SDS-PAGE. Densitometric quantification showed a total yield of 72 μg

AVITAG™-PEX2 per milliliter of RTS®500 extract. The proportion of soluble fusion protein was 50% of the total yield. The degree of biotinylation was analyzed as described in material and methods and found to be quantitative. Detection of biotinylated protein with streptavidin-POD conjugate showed no other biotinylated protein in the extract. After the affinity purification procedure using monomeric avidin, only biotinylated AVITAG™-PEX2 fusion protein was detected in the elution fractions. The identity of the fusion protein was confirmed by N-terminal degradation (Edman). Purified AVITAG™-PEX2 fusion protein as well as supernatant from dialyzed and cleared RTS®500 extract were analyzed in surface plasmon resonance spectroscopy. 105 RU purified AVITAG™-PEX2 fusion protein were attached to a BIAcore SA-chip. Saturation of the immobilized AVITAG™-PEX2 ligand with the analyte TIMP2 was achieved at 64 RUmax. Thus, an analyte binding capacity of 70% (compared to 26% according to Comparison Example 4a) could be detected. After the injection of cleared supernatant of RTS®500 extract, 732 RU biotinylated protein were immobilized on the SA-chips surface. At Rmax, 341RU TIMP2 were bound, which resembles an analyte binding capacity of 53% (compared to 15% according to Comparison Example 4a). Kinetics were measured showing an equilibrium constant of the TIMP2 to PEX2 interaction of KD=1.5×10⁻¹⁰ M. The KD was determined using the numeric model described before.

### EXAMPLE 5

### Biotinylation of AVITAG™-PEX2 by Coexpression of BirA in the RTS®500

Material and Methods:

Five RTS®500 reactions were prepared according to the manufacturer's instructions. D-Biotin was adjusted to 2 μM in each reaction. 10 μg pIVEX2.3MCS containing DNA encoding AVITAG™-PEX2 (ratio 260 nm/280 nm>1.8) was added to each reaction mixture. Instead of a supplementation with recombinant BirA, as described in Example 2, pIVEX2.3MCS containing DNA encoding BirA was added at varying amounts (1 μg, 100 ng, 10 ng, 1 ng, 0 ng, ratio 260 nm/280 nm>1.8) to the reaction chambers. During protein expression the fusion protein AVITAG™-PEX2 and BirA were simultaneously expressed. The coexpression was performed (Roche Diagnostics GmbH, Penzberg, Germany) under stirring (130 rpm) for 17 h at 30° C. The RTS® lysates were centrifuged at 10.000 g for 10 min. The supernatant of each reaction was analyzed for biotinylated AVITAG™-PEX2 fusion protein using streptavidin POD Western blotting as described in Example 3.

Results:

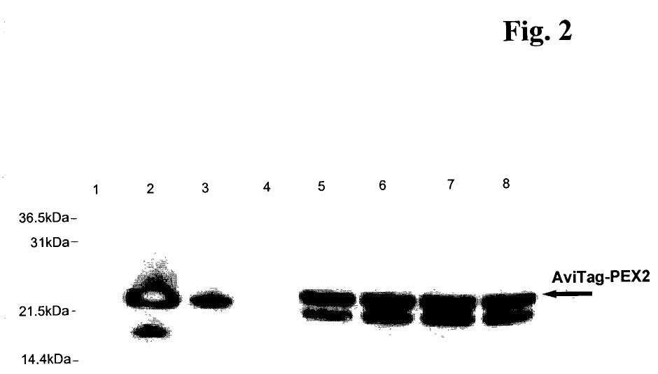

Without any supplementation of pIVEX2.3MCSBirA plasmid-DNA no biotinylated AVITAG™-PEX2 fusion protein could be detected in streptavidin POD Western blotting (FIG. 2, lane[1]), whereas addition of pIVEX2.3MCSBirA showed a biotinylated AVITAG™-PEX2 product (lanes [5,6,7,8]). 1 ng of pIVEX2.3MCSBirA plasmid-DNA inserted into the system is enough plasmid DNA to coexpress sufficient amounts of active BirA, in order to quantitatively biotinylate AVITAG™-PEX2 fusion protein.

Numerous references are cited herein, and the contents of all references cited herein are incorported by reference in their entireties.

List of References

Altman, J. D., et al., Science 274 (1996) 94-96

Brinkmann, U., et al., Gene 85 (1989) 109-114

Brooks, P. C., et al., Cell 92 (1998) 391-400

Chapman-Smith, A., and Cronan, J. E., Jr., J. Nutr. 129, 2S Suppl., (1999) 477S-484S

Cronan, J. E., Jr., Cell 58 (1989) 427-429

Cronan, J. E., Jr., et al., J. Biol. Chem. 265 (1990) 10327-10333

EP 0 932 664

Fresno, M., et al., Eur. J. Biochem. 68 (1976) 355-364

Kohanski, R. A. and Lane, M. D. (1990) Methods Enzymol. 194-200

Morag, E., et al., Anal. Biochem. 243 (1996) 257-263

Parrott, M. B., and Barry, M. A., Biochem. Biophys. Res. Communications 281 (2001) 993-1000

Pelham, H. R., and Jackson, R. J., Eur. J. Biochem. 67 (1976) 247-256

Pratt et al., Transcription and Translation: A Practical Approach, Hames and Higgins (eds.), pp. 179-209, IRL Press, 1984

Pratt, J. M., et al., Nucleic Acids Research 9 (1981) 4459-4479

Samols, D., et al., J. Biol. Chem. 263 (1988) 6461-6464

Sano, T., and Cantor, C. R., Proc. Natl. Acad. Sci.USA 92 (1995) 3180-3184

Saviranta, P., et al., Bioconjug. Chem. 9 (1998) 725-735

Schatz, P. J., Biotechnology 11 (1993) 1138-1143

Skup, D., and Millward, S., Nucleic Acids Research 4 (1977) 3581-3587

Spirin, A. S., et al., Science 242 (1988) 1162-1164

Tsao, K.-L., et al., Gene 169 (1996) 59-64

U.S. Pat. No. 5,478,730

U.S. Pat. No. 5,571,690

U.S. Pat. No. 5,723,584

U.S. Pat. No. 5,874,239

U.S. Pat. No. 5,932,433

U.S. Pat. No. 6,265,552

WO 00/55353

WO 00/58493

WO 95/04069

WO 98/31827

WO 99/37785

WO 99/50436

Yu, A. E., et al., Biochem. Cell Biol. 74 (1996) 823-831

Zubay, G., Ann. Rev. Genetics 7 (1973) 267-287

We claim:

1. A method for producing a specifically biotinylated polypeptide comprising contacting, under suitable conditions, a polypeptide to be biotinylated with a reaction mixture that comprises ribosomes, tRNA, ATP, GTP, nucleotides, biotin and amino acids, and a polypeptide that comprises an enzymatically active domain of a BirA enzyme,
wherein the polypeptide to be biotinylated comprises a BirA substrate sequence tag, and the polypeptide to be biotinylated and the polypeptide comprising an enzymatically active domain of a BirA enzyme, are expressed in situ in the reaction mixture, by at least one nucleic acid molecule encoding the polypeptide to be biotinylated, the enzymatically active domain of a BirA enzyme, respectively.

2. The method of claim 1 wherein the BirA substrate sequence tag is located at either the N-terminal or the C-terminal of the polypeptide to be biotinylated.

3. The method of claim 1 further comprising isolating the resulting specifically biotinylated polypeptide.

4. The method of claim 1 wherein the polypeptide to be biotinylated is a fusion protein comprising a polypeptide of interest and a BirA substrate sequence tag.

5. The method of claim 1 wherein the reaction mixture is a cell-free composition comprising a ribosome-containing cell lysate of a prokaryotic or eukaryotic cell.

6. The method of claim 5 wherein the reaction mixture is a cell-free composition comprising a ribosome-containing cell lysate of Escherichia coli.

7. The method of claim 1 wherein the protein comprising an enzymatically active domain of a BirA enzyme, is present in the reaction mixture in a concentration of about 10,000 to about 15,000 units per ml of reaction media.

8. The method of claim 1 wherein the polypeptide to be biotinylated has a molecular weight of about 8 kDa to about 120 kDa.

9. The method of claim 1 wherein the polypeptide to be biotinylated comprises about 100 to about 400 amino acid residues.

10. The method of claim 1 wherein the BirA substrate sequence tag is a polypeptide molecule comprising an Ala Met Lys Met motif (SEQ ID NO: 14).

11. The method of claim 1 wherein the polypeptide to be biotinylated comprises a BirA substrate sequence tag having a peptide sequence selected from the group consisting of SEQ ID NO. 1, SEQ ID NO: 2, SEQ ID NO: 3, SEQ ID NO: 4, SEQ ID NO: 5, SEQ ID NO: 6 and SEQ ID NO: 7.

12. The method of claim 1 wherein the BirA substrate sequence tag is encoded by a vector comprising an AVITAG™ encoding nucleic acid.

13. The method of claim 1 wherein the BirA substrate sequence tag is encoded by a vector comprising a PINPOINT™ encoding nucleic acid.

14. The method of claim 1 that is conducted at a temperature from about 20° C. to to about 36° C.

15. The method of claim 14 that requires from about 10 to to about 30 hours to produce a desired quantity of biotinylated protein.

16. The method of claim 1 that further comprising a step of contacting the biotinylated polypeptide to a surface that comprises a biotin binding reagent.

17. The method of claim 16 further wherein the biotin binding reagent is selected from the group consisting of avidin and streptavidin.

18. The method of claim 1 that further comprises a step of concentrating the reaction mixture by dialysis.

19. The method of claim 1 wherein the protein comprising an enzymatically active domain of a BirA enzyme is a product of the qrA gene.


SEQUENCE LISTING (14 sequences)

SEQ ID NO: 1 - protein, 123 aa, Artificial Sequence
  Description of Artificial Sequence 1.3S transcarboxylase subunit of Propionibacterium shermanii
MKLKVTVNGTAYDVDVDVDKSHENPMGTILFGGGTGGAPAPRAAGGAGAGKAGEGEIPAPLAGTVSKILVKEGDTVKAGQTVLVLEAMKMETEINAPTDGKVEKVLVKERDAVQGGQGLIKIG

SEQ ID NO: 2 - protein, 156 aa, Escherichia coli
MDIRKIKKLIELVEESGISELEISEGEESVRISRAAPAASFPVMQQAYAAPMMQQPAQSNAAAPATVPSMEAPAAAEISGHIVRSPMVGTFYRTPSPDAKAFIEVGQKVNVGDTLCIVEAMKMMNQIEADKSGTVKAILVESGQPVEFDEPLVVIE

SEQ ID NO: 3 - protein, 22 aa, Artificial Sequence
  Description of Artificial Sequence Biotinylation peptide originating from the 1.3S transcarboxylase subunit of Propionibacterium shermanii
GQTVLVLEAMKMETEINAPTDG

SEQ ID NO: 4 - protein, 21 aa, Artificial Sequence
  Description of Artificial Sequence Biotinylation peptide AAW46671
DEELNQIFEAMKMYPLVHVTK

SEQ ID NO: 5 - protein, 15 aa, Artificial Sequence
  Description of Artificial Sequence Biotinylation peptide AAW46656
LLRTFEAMKMDWRNG

SEQ ID NO: 6 - protein, 15 aa, Artificial Sequence
  Description of Artificial Sequence AviTag Biotinylation peptide
GLNDIFEAQKIEWHE

SEQ ID NO: 7 - protein, 133 aa, Artificial Sequence
  Description of Artificial Sequence PinPoint Biotinylation peptide
MKLKVTVNGTAYDVDVDVDKSHENPMGTILFGGGTGGAPAPAAGGAGAGKAGEGEIPAPLAGTVSKILVKEGDTVKAGQTVLVLEAMKMETEINAPTDGKVEKVLVKERDAVQGGQGLIKIGDLELIEGREKL

SEQ ID NO: 8 - DNA, 39 bp, Artificial Sequence
  Description of Artificial Sequence primer
ataagaataagcttcctgaaatctgcaaacaggatatcg

SEQ ID NO: 9 - DNA, 35 bp, Artificial Sequence
  Description of Artificial Sequence primer
atagtttagcggccgcttatcagcctagccagtcg

SEQ ID NO: 10 - DNA, 21 bp, Artificial Sequence
  Description of Artificial Sequence primer
gaaggcatatgggtctgaacg

SEQ ID NO: 11 - DNA, 47 bp, Artificial Sequence
  Description of Artificial Sequence primer
ctcagaaaatcgaatggcacgaagcgaccctgaaatctgcaaacagg

SEQ ID NO: 12 - DNA, 48 bp, Artificial Sequence
  Description of Artificial Sequence primer
gccattcgattttctgagcttcgaagatgtcgttcagacccatatgcc

SEQ ID NO: 13 - DNA, 30 bp, Artificial Sequence
  Description of Artificial Sequence primer
gccgctcgagtcagcagcctagccagtcgg

SEQ ID NO: 14 - protein, 4 aa, Artificial Sequence
  Description of Artificial Sequence Synthetic motif
AMKM